(19) United States
(12) Patent Application Publication
(10) Pub. No.: US 2007/0021380 A1
(43) Pub. Date: Jan. 25, 2007

(54) Novel drug delivery compositions

(76) Inventors: Leonard Wiebe, Edmonton (CA); James Diakur, Winnipeg (CA)

Correspondence Address: Craig K. Sherbume, #236, 6030-88th Street, Edmonton, AB, T6E 6G4, CA

(21) Appl. No.: 11/491,262

(22) Filed: Jul. 24, 2006

Related U.S. Application Data
(60) Provisional application No. 60/701,489, filed on Jul. 22, 2005.

(51) Int. Cl. A61K 31/724; C08B 37/00

(52) U.S. Cl. 514/58; 536/54

(57) ABSTRACT

The present invention provides for a novel molecules useful for delivery of compounds to a mammal, more particularly for the intracellular delivery of nucleotides, nucleotide analogues or compounds with a heterocyclic base. Also provided for are novel therapeutic complexes comprising novel molecules complexed with nucleotide analogues or heterogeneous or homogenous oligomers comprised of nucleotide analogues.

### RELATED APPLICATION

This application claims the benefit of U.S. Provisional Application Ser. No. 60/701,489, filed Jul. 22, 2006, under 35 U.S.C. 119(e). The entire disclosure of the prior application is hereby incorporated by reference.

### FIELD OF THE INVENTION

The present invention relates to the field of drug delivery, in particular the intracellular delivery of drugs and other compositions in vivo.

### BACKGROUND OF THE INVENTION

All of the publications, patents and patent applications cited within this application are herein incorporated by reference in their entirety to the same extent as if the disclosure of each individual publication, patent application or patent was specifically and individually indicated to be incorporated by reference in its entirety.

It is anticipated that the nucleic acid (NA) therapeutic market will reach over $210 billion in the coming years, illustrating a solid and potentially very profitable approach to treatment of cancer, rheumatoid arthritis, cardiovascular conditions. Whilst the market for NA products used as therapeutics is still at an early stage, the NA therapeutic market will live up to the expectations, unlike many other technologies that have tended to suffer from ‘hype’ than reality in the early stages.

Cyclodextrins (CyDs) are a family of cyclic oligosaccharides possessing traits suitable for application as drug carriers. These molecules consist of α(1→4) linked glucose units, thus are non-toxic, and due to their intrinsic cyclic nature, are metabolized slowly in vivo. In terms of molecular structure, CyDs can be viewed as molecular buckets. The cavity sizes for the commonly available α-, β- and γ-CyDs are 4.9, 6.2 and 7.9 angstroms respectively, and these dimensions are ideal for the inclusion of low molecular weight lipophilic drugs. Their ability to form inclusion complexes has been exploited for altering the chemical and physical properties of guest (drug) molecules including solubility, in vivo stability, reduction of toxicity and irritancy, and improved bioavailability. As release is governed by dissociation, it is anticipated that selective chemical modifications can be employed to optimize equilibrium thermodynamics, and thus release. Along with a relatively hydrophobic interior cavity for the molecular encapsulation of guest molecules, the architecture of CyDs is such that two faces are apparent. The immediate consequence of this spatial arrangement is that the two faces can be exploited for separate functions (ie. therapeutic and targeting functions).

Since their first introduction in 1970, liposomes have been extensively studied for potential applications as drug delivery agents in medicine, immunology, diagnostics, and cosmetics. Due to their colloidal and biological instability, as well as their inefficient and unstable encapsulation of drug molecules, their widespread application in medicine to date, has been limited. Just recently, the FDA recommended not to approve Evacet™ (a doxorubicin liposome formulation) for the treatment of metastatic breast cancer. Doxorubicin is active against a broad spectrum of solid tumors, however, is associated with undesirable cardiac toxicity. Overall, liposomes are subject to many other types of biodegradation processes, thus there is room for the development of alternative carriers which incorporate the favourable properties of liposomes. Cyclodextrin formulations potentially represent a new approach to carrying molecules in circulation while avoiding degradation processes. It is therefore an object of the present invention to provide novel compounds for improved intracellular delivery of compounds to a mammal.

### SUMMARY OF THE INVENTION

In one aspect, the present invention provides novel molecules useful for delivery of compounds to a mammal comprising compounds of the formula:

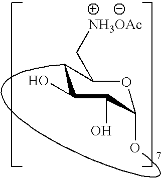

In another aspect, the present invention provides novel molecules useful for delivery of compounds to a mammal comprising compounds of the formula

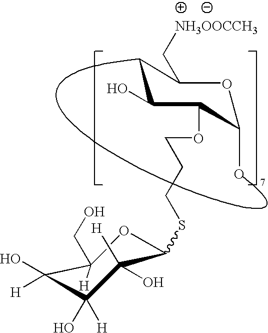

In a further aspect the present invention provides for novel molecules useful for delivery of compounds to a mammal comprising compounds of the general formula: 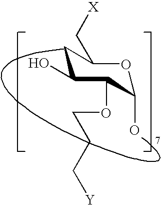
Wherein X may be a molecule of net positive or negative charge at physiologic conditions, more preferably a molecule containing at least one amino or at least one amide group; and wherein Y may be a carbohydrate, peptide, or protein which results in the enhancing of intracellular delivery of the compounds of the present invention to cells in general, or through the selected and/or preferential delivery to a cell sub-type. It is contemplated that X may be partially substituted within the cylcodextrin ring structure; wherein at least one of the heptomeric subunits is substituted at the position represented by X with a molecule of net positive or negative charge at physiologic conditions. It is contemplated that Y may be a carbohydrate structure bound by receptors located on sub-populations of cells in a mammal; peptides bound by receptors located on sub-populations of cells in a mammal; or proteins, such as antibodies, which bind preferentially to antigens located on sub-populations of cells in a mammal. In a preferred embodiment Y is a galactose.

In a further aspect, the present invention provides for molecules capable of increasing the delivery and/or efficacy of compounds for administration to a mammal. More particularly, the molecules disclosed herein may be utilized to increase the delivery of compounds to the intracellular environment, for example compounds of a net ionic charge not otherwise easily translocated across the membrane of a mammalian cell. In a preferred embodiment, an effective amount of the molecules of the present invention are combined with an effective amount of therapeutic agent for treatment of a disease through introduction to a mammalian system or intracellular environment.

In another aspect the present invention provides for novel therapeutic for viral infection in a mammal comprising compounds of the general formula: 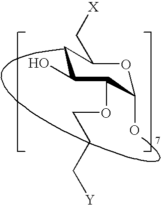
complexed with a compound of the general formula
Bₙ
Wherein B is a polynucleotide analogue of compound with a heterocyclic base, formed by phosphodiester bonds of size n=2,3,4, . . . 50. In a preferred embodiment n=8-20, and in a more preferred embodiment n=12. The present invention contemplates the use of any therapeutically useful nucleotide analogue (a phosphorylated nucleoside analogue), either as a homogenous polynucleotide or in combination with at least one other nucleoside analogue. One skilled in the art would be capable of identifying nucleotide analogues therapeutically useful in treatment of virally infected cells. Such nucleotide analogues include, but are not limited to homogenous or heterogeneous phosphorylated oligomers of Abacavir, 2′-3′-dideoxyinosine, ddI, 2′,3′-dideoxy-3′-thiacytidine (3TC), Emtricitabine (FTC), Stavudine, Zalcitabine, azidothymidine (AZT), Ganciclovir, Valganciclovir, Cytarabine, Edoxudin, Ribavirin, Idoxuridine, AIdUrd, Bromodeoxyuridine, ara-T, Fiacitabine, Brivudine, 9-(2,3-dihydroxypropyl)adenine, Deoxyuridine, and Tenofovir Disoproxil Fumarate (TDF). It is contemplated that X may be partially substituted within the cylcodextrin ring structure; wherein at least one of the heptomeric subunits is substituted at the position represented by X with a molecule of net positive or negative charge at physiologic conditions. It is contemplated that Y may be a carbohydrate structure bound by receptors located on sub-populations of cells in a mammal; peptides bound by receptors located on sub-populations of cells in a mammal; or proteins, such as antibodies, which bind preferentially to antigens located on sub-populations of cells in a mammal. In a preferred embodiment Y is a galactose.

In another aspect the present invention provides for novel therapeutic for viral infection in a mammal comprising compounds of the general formula: 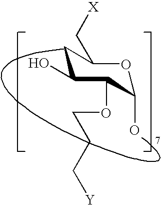
complexed with a compound of the general formula
C

wherein C is a nucleotide analogue or compound with a heterocyclic base. One skilled in the art would be capable of identifying nucleotide analogues therapeutically useful in treatment of virally infected cells. Such nucleotide analogues include, but are not limited to Abacavir, 2′-3′-dideoxyinosine, ddI, 2′,3′-dideoxy-3′-thiacytidine (3TC), Emtricitabine (FTC), Stavudine, Zalcitabine, azidothymidine (AZT), Ganciclovir, Valganciclovir, Cytarabine, Edoxudin, Ribavirin, Idoxuridine, AIdUrd, Bromodeoxyuridine, ara-T, Fiacitabine, Brivudine, 9-(2,3-dihydroxypropyl)adenine, Deoxyuridine, and Tenofovir Disoproxil Fumarate (TDF). It is contemplated that X may be partially substituted within the cylcodextrin ring structure; wherein at least one of the heptomeric subunits is substituted at the position represented by X with a molecule of net positive or negative charge at physiologic conditions. It is contemplated that Y may be a carbohydrate structure bound by receptors located on sub-populations of cells in a mammal; peptides bound by receptors located on sub-populations of cells in a mammal; or proteins, such as antibodies, which bind preferentially to antigens located on sub-populations of cells in a mammal. In a preferred embodiment Y is a galactose.

In another aspect the present invention provides for the use of compounds of the general formula: 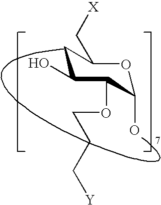
for intracellular delivery of small inhibitory RNA (siRNA) or other oligomeric nucleotides or deoxynucleotides for therapeutic effect. In a further embodiment the compounds of the present invention are used for protection and/or increasing serum half-life of siRNA or other oligonucleotide or deoxynucleotide in a mammal.

The accompanying description illustrates preferred embodiments of the present invention and serves to explain the principles of the present invention.

### BRIEF DESCRIPTION OF THE DRAWINGS

FIG. 1 shows the standard curve of AMP in water in QNMR analysis;

FIG. 2 shows the QNMR spectra of a mixture of AMP/BCD 1:1 mole ratio (20 mg), dibasic Potassium phosphate (2.5 mg) and TEP(3 mg) in water(0.5 mL),

FIG. 3 shows the QNMR spectra of a mixture of AMP/BCD 1:1 mole ratio (20 mg), dibasic Potassium phosphate (2.5 mg) and TEP(3 mg) in water(0.5 mL) analysed using a Line Fitting Method;

FIG. 4 shows the QNMR spectra of a mixture of AMP/ABCDA 1:1 mole ratio (26 mg), Potassium phosphate (2.5 mg) and TEP(2.5 mg) in water(0.5 mL);

FIG. 5 shows the QNMR spectra of a mixture of AMP/ABCDA 1:1 mole ratio (26 mg), Potassium phosphate (2.5 mg) and TEP(2.5 mg) in water(0.5 mL) analysed using a Line Fitting Method;

FIG. 6 shows the QNMR spectra of a mixture of AMP/ABCDA 1:1 mole ratio (13 mg), Potassium phosphate (2.5 mg) and TEP(2.5 mg) in water(0.5 mL) analysed using a Line Fitting Method;

FIG. 7 shows DSC thermograms for the AMP/BCD inclusion complex;

FIG. 8 shows DSC thermograms for the AMP/ABCDA inclusion complex.

### DETAILED DESCRIPTION OF THE INVENTION

As used herein, “administration” means the introduction of a compound to a mammal, either systemically or localized to an organ or tissue, through means generally known in the art, such that the administered compound is capable of interacting with the general tissue or organ, or cells of interest. Examples of such means generally known in the art include, but are not limited to, oral formulations, intravenous injection, catheterization, suppository, and direct introduction to a tissue through injection.

As used herein “disease” means a state in a mammal which may directly or indirectly lead to a cellular, tissue, organ or systemic state detrimental to the mammal.

As used herein and “effective amount” is an amount of a sufficient to achieve the intended purpose. The effective amount of a given therapeutic agent will vary with factors such as the nature of the agent, the route of administration, the size and species of the animal to receive the therapeutic agent, and the purpose of the administration. The effective amount of a molecule of the present invention will depend upon the compound with which it will be complexed, temperature of the combined molecule and compound, pH of the solution with which they exist, and the presence of ionic salts in the solution. The effective amount in each individual case may be determined by a skilled artisan according to established methods in the art.

As used herein “nucleotide” corresponds to both oxy-, and deoxyribonucleotides.

As used herein a “therapeutic compound contemplated by the present invention” is a compound described generally as:
Bₙ or C
Wherein B is a polynucleotide analogue of compound with a heterocyclic base, formed by phosphodiester bonds of size n=2,3,4, . . . 50. In a preferred embodiment n=8-20, and in a more preferred embodiment n=12. The present invention contemplates the use of any therapeutically useful nucleotide analogue (a phosphorylated nucleoside analogue), either as a homogenous polynucleotide or in combination with at least one other nucleoside analogue; and
Wherein C is a nucleotide analogue or compound with a heterocyclic base.

One skilled in the art would be capable of identifying nucleotide analogues therapeutically useful in treatment of virally infected cells. Such nucleotide analogues include, but are not limited to Abacavir, 2′-3′-dideoxyinosine, ddI, 2′,3′-dideoxy-3′-thiacytidine (3TC), Emtricitabine (FTC), Stavudine, Zalcitabine, azidothymidine (AZT), Ganciclovir, Valganciclovir, Cytarabine, Edoxudin, Ribavirin, Idoxuridine, AIdUrd, Bromodeoxyuridine, ara-T, Fiacitabine, Brivudine, 9-(2,3-dihydroxypropyl)adenine, Deoxyuridine, and Tenofovir Disoproxil Fumarate (TDF).

The present invention provides for molecules useful administration of compounds to a mammal generally or for the intracellular delivery of compounds. As used herein a “compound of the present invention” is of the general formula: 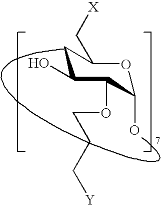
Wherein X may be a compound of net positive or negative charge at physiologic conditions, more preferably a molecule containing at least one amino or at least one amide group; and wherein Y may be a carbohydrate, peptide, or protein which results in the enhancing of intracellular delivery of the compounds of the present invention. It is contemplated that X may be partially substituted within the cylcodextrin ring structure; wherein at least one of the heptomeric subunits is substituted at the position represented by X with a molecule of net positive or negative charge at physiologic conditions. It is contemplated that Y may be a carbohydrate structure bound by receptors located on sub-populations of cells in a mammal; peptides bound by receptors located on sub-populations of cells in a mammal; or proteins, such as antibodies, which bind preferentially to antigens located on sub-populations of cells in a mammal. In a preferred embodiment Y is a galactose.

More particularly, the present invention provides for molecules of the formula

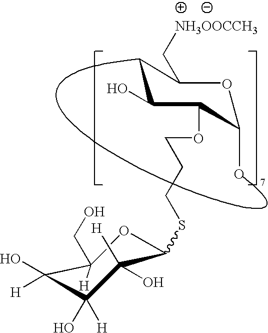

More particularly, the present invention provides for molecules of the formula

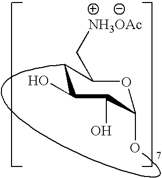

It is appreciated that compounds of the present invention may have a chiral center and may exist in and be isolated in optically active and racemic forms. Some compounds may exhibit polymorphism. It is to be understood that the present invention encompasses any racemic, optically-active, diastereomeric, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

Examples of methods to obtain optically active materials are known in the art, and include at least the following.

i) physical separation of crystals—a technique whereby macroscopic crystals of the individual enantiomers are manually separated. This technique can be used if crystals of the separate enantiomers exist, i.e., the material is a conglomerate, and the crystals are visually distinct;

ii) simultaneous crystallization—a technique whereby the individual enantiomers are separately crystallized from a solution of the racemate, possible only if the latter is a conglomerate in the solid state;

iii) enzymatic resolutions—a technique whereby partial or complete separation of a racemate by virtue of differing rates of reaction for the enantiomers with an enzyme;

iv) enzymatic asymmetric synthesis—a synthetic technique whereby at least one step of the synthesis uses an enzymatic reaction to obtain an enantiomerically pure or enriched synthetic precursor of the desired enantiomer;

v) chemical asymmetric synthesis—a synthetic technique whereby the desired enantiomer is synthesized from an achiral precursor under conditions that produce asymmetry (i.e., chirality) in the product, which may be achieved using chiral catalysts or chiral auxiliaries;

vi) diastereomer separations—a technique whereby a racemic compound is reacted with an enantiomerically pure reagent (the chiral auxiliary) that converts the individual enantiomers to diastereomers. The resulting diastereomers are then separated by chromatography or crystallization by virtue of their now more distinct structural differences and the chiral auxiliary later removed to obtain the desired enantiomer;

vii) first- and second-order asymmetric transformations—a technique whereby diastereomers from the racemate equilibrate to yield a preponderance in solution of the diastereomer from the desired enantiomer or where preferential crystallization of the diastereomer from the desired enantiomer perturbs the equilibrium such that eventually in principle all the material is converted to the crystalline diastereomer from the desired enantiomer. The desired enantiomer is then released from the diastereomer;

viii) kinetic resolutions—this technique refers to the achievement of partial or complete resolution of a racemate (or of a further resolution of a partially resolved compound) by virtue of unequal reaction rates of the enantiomers with a chiral, non-racemic reagent or catalyst under kinetic conditions;

ix) enantiospecific synthesis from non-racemic precursors—a synthetic technique whereby the desired enantiomer is obtained from non-chiral starting materials and where the stereochemical integrity is not or is only minimally compromised over the course of the synthesis;

x) chiral liquid chromatography—a technique whereby the enantiomers of a racemate are separated in a liquid mobile phase by virtue of their differing interactions with a stationary phase. The stationary phase can be made of chiral material or the mobile phase can contain an additional chiral material to provoke the differing interactions;

xi) chiral gas chromatography—a technique whereby the racemate is volatilized and enantiomers are separated by virtue of their differing interactions in the gaseous mobile phase with a column containing a fixed non-racemic chiral adsorbent phase;

xii) extraction with chiral solvents—a technique whereby the enantiomers are separated by virtue of preferential dissolution of one enantiomer into a particular chiral solvent;

xiii) transport across chiral membranes—a technique whereby a racemate is placed in contact with a thin membrane barrier. The barrier typically separates two miscible fluids, one containing the racemate, and a driving force such as concentration or pressure differential causes preferential transport across the membrane barrier. Separation occurs as a result of the non-racemic chiral nature of the membrane which allows only one enantiomer of the racemate to pass through.

Pharmaceutically Acceptable Salt

In cases where compounds are sufficiently basic or acidic to form stable nontoxic acid or base salts, administration of the compound as a pharmaceutically acceptable salt may be appropriate. The term “pharmaceutically acceptable salts” or “complexes” refers to salts or complexes that retain the desired biological activity of the compounds of the present invention and exhibit minimal undesired toxicological effects.

Examples of pharmaceutically acceptable salts are organic acid addition salts formed with acids, which form a physiological acceptable anion, for example, tosylate, methanesulfonate, acetate, citrate, malonate, tartarate, succinate, benzoate, ascorbate, alpha.-ketoglutarate and .alpha.-glycerophosphate. Suitable inorganic salts may also be formed, including, sulfate, nitrate, bicarbonate and carbonate salts. Alternatively, the pharmaceutically acceptable salts may be made with sufficiently basic compounds such as an amine with a suitable acid affording a physiologically acceptable anion. Alkali metal (for example, sodium, potassium or lithium) or alkaline earth metal (for example calcium) salts of carboxylic acids can also be made.

Nonlimiting examples of such salts are (a) acid addition salts formed with inorganic acids (for example, hydrochloric acid, hydrobromic acid, sulfuric acid, phosphoric acid, nitric acid, and the like), and salts formed with organic acids such as acetic acid, oxalic acid, tartaric acid, succinic acid, malic acid, ascorbic acid, benzoic acid, tannic acid, pamoic acid, alginic acid, polyglutamic acid, naphthalenesulfonic acid, naphthalenedisulfonic acid, and polygalcturonic acid; (b) base addition salts formed with metal cations such as zinc, calcium, bismuth, barium, magnesium, aluminum, copper, cobalt, nickel, cadmium, sodium, potassium, and the like, or with a cation formed from ammonia, N,N-dibenzylethylenediamine, D-glucosamine, tetraethylammonium, or ethylenediamine; or (c) combinations of (a) and (b); e.g., a zinc tannate salt or the like.

Particular FDA-approved salts can be conveniently divided between anions and cations (Approved Drug Products with Therapeutic Equivalence Evaluations (1994) U.S. Department of Health and Human Services, Public Health Service, FDA, Center for Drug Evaluation and Research, Rockville, Md.; L. D. Bighley, S. M. Berge and D. C. Monkhouse, Salt Forms of Drugs and Absorption, Encyclopedia of Pharmaceutical Technology, Vol. 13, J. Swarbridk and J. Boylan, eds., Marcel Dekker, NY (1996)). Among the approved anions include aceglumate, acephyllinate, acetamidobenzoate, acetate, acetylasparaginate, acetylaspartate, adipate, aminosalicylate, anhydromethylenecitrate, ascorbate, aspartate, benzoate, besylate, bicarbonate, bisulfate, bitartrate, borate, bromide, camphorate, camsylate, carbonate, chloride, chlorophenoxyacetate, citrate,closylate, cromesilate, cyclamate, dehydrocholate, dihydrochloride, dimalonate, edentate, edisylate, estolate, esylate, ethylbromide, ethylsulfate, fendizoate, fosfatex, fumarate, gluceptate, gluconate, glucuronate, glutamate, glycerophosphate, glysinate, glycollylarsinilate, glycyrrhizate, hippurate, hemisulfate, hexylresorcinate, hybenzate, hydrobromide, hydrochloride, hydroiodid, hydroxybenzenesulfonate, hydroxybenzoate, hydroxynaphthoate, hyclate, iodide, isethionate, lactate, lactobionate, lysine, malate, maleate, mesylate, methylbromide, methyliodide, methylnitrate, methylsulfate, monophosadenine, mucate, napadisylate, napsylate, nicotinate, nitrate, oleate, orotate, oxalate, oxoglurate, pamoate, pantothenate, pectinate, phenylethylbarbiturate, phosphate, pacrate, plicrilix, polistirex, polygalacturonate, propionate, pyridoxylphosphate, saccharinate, salicylate, stearate, succinate, stearylsulfate, subacetate, succinate, sulfate, sulfosalicylate, tannate, tartrate, teprosilate, terephthalate, teoclate, thiocyante, tidiacicate, timonacicate, tosylate, triethiodide, triethiodide, undecanoate, and xinafoate. The approved cations include ammonium, benethamine, benzathine, betaine, calcium, camitine, clemizole, chlorcyclizine, choline, dibenylamine, diethanolamine, diethylamine, diethylammonium diolamine, eglumine, erbumine, ethylenediamine, heptaminol, hydrabamine, hydroxyethylpyrrolidone, imadazole, meglumine, olamine, piperazine, 4-phenylcyclohexylamine, procaine, pyridoxine, triethanolamine, and tromethamine. Metallic cations include, aluminum, bismuth, calcium lithium, magnesium, neodymium, potassium, rubidium, sodium, strontium and zinc.

A particular class of salts can be classified as organic amine salts. The organic amines used to form these salts can be primary amines, secondary amines or tertiary amines, and the substituents on the amine can be straight, branched or cyclic groups, including ringed structures formed by attachment of two or more of the amine substituents. Of particular interest are organic amines that are substituted by one or more hydroxyalkyl groups, including alditol or carbohydrate moieties. These hydroxy substituted organic amines can be cyclic or acyclic, both classes of which can be primary amines, secondary amines or tertiary amines. A common class of cyclic hydroxy substituted amines are the amino sugars.

The invention also includes pharmaceutically acceptable prodrugs of the therapeutic compound contemplated by the present invention. Pharmaceutically acceptable prodrugs refer to a compound that is metabolized, for example hydrolyzed or oxidized, in the host to form the compound of the present invention. Typical examples of prodrugs include compounds that have biologically labile protecting groups on a functional moiety of the active compound. Prodrugs include compounds that can be oxidized, reduced, aminated, deaminated, hydroxylated, dehydroxylated, hydrolyzed, dehydrolyzed, alkylated, dealkylated, acylated, deacylated, phosphorylated, dephosphorylated to produce the active compound. Any of the therapeutic compounds contemplated by the present invention can be administered as a prodrug to increase the activity, bioavailability, stability or otherwise alter the properties of the compound.

Pharmaceutical Compositions and Formulation

Any host organism, including a pateint, mammal, and specifically a human, suffering from a viral infection can be treated by the administration of a composition comprising an effective amount of the compounds of the present invention complexed with a therapeutic compound contemplated by the present invention, optionally in a pharmaceutically acceptable carrier or diluent. The complex can be administered in any desired manner, including oral, topical, parenteral, intravenous, intradermal, intra-articular, intra-synovial, intrathecal, intra-arterial, intracardiac, intramuscular, subcutaneous, intraorbital, intracapsular, intraspinal, intrasternal, topical, transdermal patch, via rectal, vaginal or urethral suppository, peritoneal, percutaneous, nasal spray, surgical implant, internal surgical paint, infusion pump, or via catheter. For standard information on pharmaceutical formulations, see Ansel, et al., Pharmaceutical Dosage Forms and Drug Delivery Systems, Sixth Edition, Williams & Wilkins (1995). Dose

An effective dose for any of the herein described conditions can be readily determined by the use of conventional techniques and by observing results obtained under analogous circumstances. In determining the effective dose, a number of factors are considered including, but not limited to: the species of patient; its size, age, and general health; the specific disease involved; the degree of involvement or the severity of the disease; the response of the individual patient; the particular compound administered; the mode of administration; the bioavailability characteristics of the preparation administered; the dose regimen selected; and the use of concomitant medication.

The concentration of the therapeutic compound contemplated by the present invention in the drug composition will depend on absorption, inactivation, and excretion rates of the drug as well as other factors known to those of skill in the art. It is to be noted that dosage values will also vary with the severity of the condition to be alleviated. It is to be further understood that for any particular subject, specific dosage regimens should be adjusted over time according to the individual need and the professional judgment of the person administering or supervising the administration of the compositions, and that the dosage ranges set forth herein are exemplary only and are not intended to limit the scope or practice of the claimed composition. The active ingredient may be administered at once, or may be divided into a number of smaller doses to be administered at varying intervals of time.

Formulation

While it may be possible for the compounds of the present invention complexed with a therapeutic compound contemplated by the present invention to be administered as the raw complex, it is preferable to present them as a pharmaceutical composition. According to a further aspect, the present invention provides a pharmaceutical composition comprising a complex of a compound of the present invention and a therapeutic compound contemplated by the present invention, together with one or more pharmaceutically acceptable carriers thereof and optionally one or more other therapeutic ingredient. The carrier(s) must be acceptable in the sense of being compatible with the other ingredients of the formulation and not deleterious to the recipient thereof.

The formulations include those suitable for oral, parenteral (including subcutaneous, intradermal, intramuscular, intravenous and intraarticular), rectal and topical (including dermal, buccal, sublingual and intraocular) administration although the most suitable route may depend upon for example the condition and disorder of the recipient.

Formulations suitable for oral administration may be presented in discrete units, such as capsules, cachets, lozenges, or tablets, each containing a predetermined amount of the compound as powder or granules; as a solution or a suspension in an aqueous or non-aqueous liquid; or as an oil-in-water or water-in-oil emulsion. As indicated, such formulations may be prepared by any suitable method of pharmacy which includes the step of bringing into association the active compound and the carrier or excipient (which may constitute one or more accessory ingredients). The carrier must be acceptable in the sense of being compatible with the other ingredients of the formulation and must not be deleterious to the recipient. The carrier may be a solid or a liquid, or both, and is preferably formulated with the compound as a unit-dose formulation, for example, a tablet, which may contain from 0.05% to 95% by weight of the active compound. Other pharmacologically active substances may also be present including other compounds. The formulations of the invention may be prepared by any of the well known techniques of pharmacy consisting essentially of admixing the components. All methods include the step of bringing into association a compound of the present invention or a pharmaceutically acceptable salt or solvate thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

For solid compositions, conventional nontoxic solid carriers include, for example, pharmaceutical grades of mannitol, lactose, starch, magnesium stearate, sodium saccharin, talc, cellulose, glucose, sucrose, magnesium carbonate, and the like. Liquid pharmacologically administrable compositions can, for example, be prepared by dissolving, dispersing, etc., an active compound as described herein and optional pharmaceutical adjuvants in an excipient, such as, for example, water, saline, aqueous dextrose, glycerol, ethanol, and the like, to thereby form a solution or suspension. In general, suitable formulations may be advantageously prepared by uniformly and intimately admixing the active compound with a liquid or finely divided solid carrier, or both, and then, if necessary, shaping the product. For example, a tablet may be prepared by compressing or molding a powder or granules of the compound, optionally with one or more accessory ingredients. Compressed tablets may be prepared by compressing, in a suitable machine, the compound in a free-flowing form, such as a powder or granules optionally mixed with a binder, lubricant, inert diluent and/or surface active/dispersing agent(s). Molded tablets may be made by molding, in a suitable machine, the powdered compound moistened with an inert liquid diluent.

Formulations suitable for buccal (sub-lingual) administration include lozenges comprising a compound in a flavored base, usually sucrose and atacia or tragacanth, and pastilles comprising the compound in an inert base such as gelatin and glycerin or sucrose and acacia.

Formulations of the present invention suitable for parenteral administration comprise sterile aqueous preparations of the compounds, which are approximately isotonic with the blood of the intended recipient. These preparations are administered intravenously, although administration may also be effected by means of subcutaneous, intramuscular, or intradermal injection. Such preparations may conveniently be prepared by admixing the compound with water and rendering the resulting solution sterile and isotonic with the blood. Injectable compositions according to the invention will generally contain from 0.1 to 5% w/w of the active compound.

Formulations suitable for rectal administration are presented as unit-dose suppositories. These may be prepared by admixing the compound with one or more conventional solid carriers, for example, cocoa butter, and then shaping the resulting mixture.

Formulations suitable for topical application to the skin preferably take the form of an ointment, cream, lotion, paste, gel, spray, aerosol, or oil. Carriers and excipients which may be used include vaseline, lanoline, polyethylene glycols, alcohols, and combinations of two or more thereof. The active compound is generally present at a concentration of from 0.1 to 15% w/w of the composition, for example, from 0.5 to 2%.

Combination and Alternation Therapy

The complex between a compound of the present invention complexed with a therapeutic compound contemplated by the present invention can be administered in combination or alternation with a second biologically active agent to increase its effectiveness against the target disorder. Any of the compounds described herein for combination or alternation therapy can be administered as any derivative that upon administration to the recipient, is capable of providing directly or indirectly, the parent compound, or that exhibits activity itself. Nonlimiting examples are the pharmaceutically acceptable salts (alternatively referred to as “physiologically acceptable salts”), and a compound which has been alkylated or acylated at an appropriate position. The modifications can affect the biological activity of the compound, in some cases increasing the activity over the parent compound.

In combination therapy, effective dosages of two or more agents are administered together, whereas during alternation therapy an effective dosage of each agent is administered serially. The dosages will depend on absorption, inactivation and excretion rates of the drug as well as other factors known to those of skill in the art. It is to be noted that dosage values will also vary with the severity of the condition to be alleviated. It is to be further understood that for any particular subject, specific dosage regimens and schedules should be adjusted over time according to the individual need and the professional judgment of the person administering or supervising the administration of the compositions.

The efficacy of a drug can be prolonged, augmented, or restored by administering the compound in combination or alternation with a second, and perhaps third, agent that induces a different biological pathway from that caused by the principle drug. Alternatively, the pharmacokinetics, biodistribution or other parameter of the drug can be altered by such combination or alternation therapy. In general, combination therapy is typically preferred over alternation therapy because it induces multiple simultaneous stresses on the condition.

Any method of alternation can be used that provides treatment to the patient. Nonlimiting examples of alternation patterns include 1-6 weeks of administration of an effective amount of one agent followed by 1-6 weeks of administration of an effective amount of a second agent. The alternation schedule can include periods of no treatment. Combination therapy generally includes the simultaneous administration of an effective ratio of dosages of two or more active agents.

Illustrative examples of specific agents that can be used in combination or alternation with the compounds of the present invention are described below. The agents set out below or others can alternatively be used to treat a host suffering from cardiovascular and inflammatory diseases:

### EXAMPLE 1

### CyD Protection

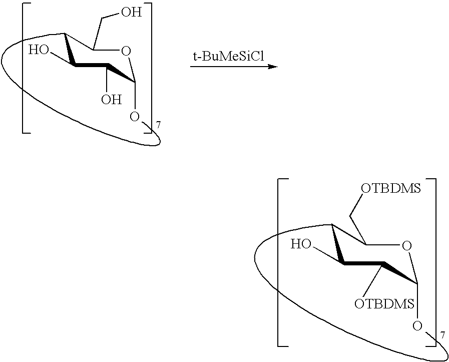

### EXAMPLE 2

### Preparation of Ethylene Glycol Ditosylate

(HOCH₂)₂→(TsOCH₂)₂

### EXAMPLE 3

### The Synthesis of Thioacetamide

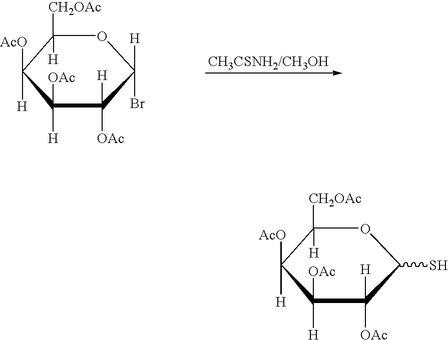

### EXAMPLE 4

### 2-Galactosyl-6-AminoCyD Synthesis

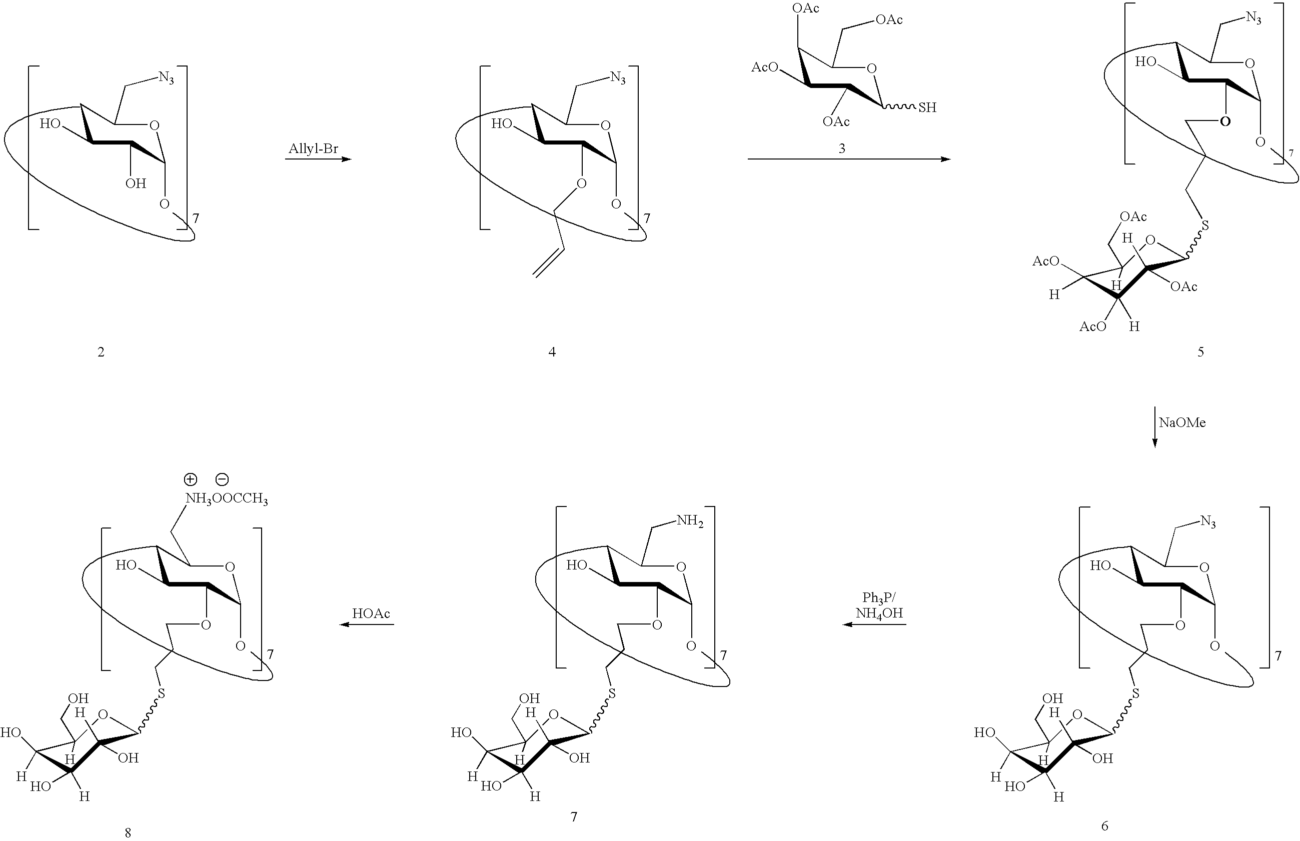

β-Cyclodextrin (β-CyD) was purchased from Sigma-Aldrich Chemical Company Inc., and was dried over P₂0₅ at room temperature for 24-48 h before use. Other chemicals were purchased from Sigma-Aldrich and Fisher. Solvents were purchased from Sigma-Aldrich and dried according to literature procedures. The solvents used for synthesis were reagent grade unless specified and other chemicals and solvents were of analytical grade.

Thin-layer chromatography (TLC) was carried out on Kieselgel 60 F254 (Merck) and visualization was accomplished by charring with 5% methanolic sulfuric acid. Column chromatography was performed using silica gel 60 (70-230 Mesh ASTM, 0.063-0.2 mm, Rose Scientific Ltd.). A Labconco Freeze Dryer 3 was used for lyophilization. IR spectra were recorded with a Perkin-Elmer 700 spectrometer. ¹H-NMR spectra and ¹³C-NMR spectra were recorded at Bruker AM-300 (75 MHz for carbon). All chemical shifts are quoted in ppm, referenced to residual CHCl₃ at δ 7.27 for CDCl₃ solutions or HOD at 8 4.82 (25° C.) for D₂O solutions. Coupling constants (J) are reported in Hertz. ¹³C-NMR spectral assignments were aided by the J-MOD technique.

Heptakis(6-bromo-6-deoxy)-β-cyclodextrin (1).

A solution of bromine (8.35 mL, 162 mmol) and DMF (35 mL) was added slowly to a solution of triphenylphosphine (42.5 g, 162 mmol) and DMF (125 mL) which was cooled (ice-bath) and stirred. After 30 min, dry β-cyclodextrin (8.75 g, 7.7 mmol) was added and the reaction mixture was stirred at 80° C. for 15 h. The mixture was concentrated to half the volume under high vacuum. The pH was adjusted to 9-10 by fresh CH₃ONa/CH₃OH (3 M, 130 mL) with simultaneous cooling. The solution was allowed to be at room temperature for 30 min and poured into ice water. The precipitate was collected by filtration and washed sequentially with water, ether and dichloromethane and dried over night in vacuo. Yield 11.98 g (quantitative).

Heptakis(6-azido-6-deoxy)-β-cyclodextrin (2).

A solution of 1 (11.98 g, 7.6 mmol) and NaN₃ (10.38 g, 160 mmol) in DMF (140 mL) was stirred at 90° C. After 60 h, the reaction mixture was allowed to cool and diluted with DMF, then evaporated until 25% of the solution remained. Water (200 mL) was added, the suspension filtered, and the solid was collected and washed with water, CH2CI2 and ether. The residual brown solid was dried overnight to yield 4.34 g (85%) of 2. ¹H-NMR (300 MHz, DMSO-d₆): δ 5.74 (s, 1H), 4.88-4.87 (d, 4H), 3.76-3.68 (m, 2H), 3.37-3.29 (m, 2H); ¹³C-NMR (75 MHz, DMSO-d₆): δ 102.1 (C-1), 83.2 (C-4), 72.6, 72.1, 70.4 (C-2, 3, 5), 51.4 (C-6).

2,3,4,6-Tetra-O-acetyl-1-thio-β-D-galactopyranose (3).

Thioacetamide(0.4 g, 5.4 mmol) was mixed with 2,3,4,6-tetra-O-acetyl-α-D-galacopyranosyl bromide (2.1 g, 5 mmol), and heated 120° C. under an atmosphere of Ar for 10 min. The reaction mixture was allowed to cool and methanol (40 ml) was added to dissolve the solid. Solvents were removed in vacuo, the residue was purified by column chromatograph using hexane-ethyl acetate(1:1) as eluents to give 3 (1.33 g, 73%); ¹H-NMR (CDCl₃, 300 MHz): δ 1.98, 2.04, 2.05, 2.09 (12H, 4s, 4Ac), 2.37 (1H, d, J=10.1 Hz, SH), 3.92-3.97 (1H, m, H-5), 4.03-4.17 (1H, m, H-6a), 4.51-4.63 (1H, m, H-6b), 4.54 (1H, t, H-1), 4.99-5.03 (1H, dd, J=10.1, H-2), 5.15-5.30 (2H, m, H-4 and H-3); ¹³C-NMR (CDCl₃, 75 MHz): δ 20.6, 20.7, 20.7, 20.8, 61.5, 67.2, 71.5, 74.9, 76.7, 79.1, 169.7, 169.9, 170.0, 170.9; ES-MS m/z: 387.1 [M +Na]⁺.

Heptakis(6-azido-6-deoxy-2-O-allyl)-β-cyclodextrin(4).

NaH powder (60%, 1.27 g, 31.63 mmol) was added to a solution of 2 (5.18 g, 3.95 mmol) in DMF (240 mL) under an atmosphere of Ar at 0° C. The reaction mixture was allowed to stir at 0° C. for 1.5 h and overnight at room temperature. Allyl bromide (3.87 g, 32 mmol) was added dropwise, and stirred at 0° C. for 1 h and overnight at room temperature. The solution was poured into ice water. The precipitate was collected by filtration and purified by column chromatograph using hexane-ethyl acetate (1:1) as eluents to give 3 (2.56 g, 40% ) as a white solid. ¹H-NMR (CDCl₃, 300 MHz): δ 3.31 (7H, t, H-2), 3.42-3.47 (7H, dd, H-4), 3.52-3.59 (7H, dd, H-5), 3.71-3.75 (14H, m, H-6), 3.88-3.94 (7H, t, H-3), 4.21-4.27 (7H, dd, OCHₐ), 4.45-4.51 (7H, dd, OCH_{b}), 4.83 (7H, d, H-1), 4.91 (7H, s, OH), δ 5.24-5.35 (14H, m, CH═CH₂), 5.87-6.00 (7H, m, CH═CH₂); ¹³C-NMR (CDCl₃, 75 MHz): δ 51.5 (C-6), 70.2, 73.0, 78.7, 84.7(C-2, C-3, C-4, C-5), 73.6 (OCH2), 101.8 (C-1), 119.2 (CH₂═CH), 134.3 (CH₂═CH); MALDI m/z: 1613 [M+Na]⁺; Anal.: Calcd for C₆₃H₉₁N₂₁O₂₈; C, 47.57; H, 5.77; N, 18.49. Found: C, 47.65; H, 5.66; N, 18.19.

Heptakis[6-azido-6-deoxy-2-O-(3-(2′,3′,4′,6′-tetra-O-acetyl-β-D-thiogalactopyranosyl)propyl)]-β-cyclodextrin(5).

3 (280 mg, 0.77 mmol) and 4 (100 mg, 0.063 mmol ) was dissolved in MeCN (40 mL). AIBN (30 mg) was added under an atmosphere of Ar at 70° C. After 7 h, the solvent was removed in vacuo, and the residue was purified by column chromatograph using hexane-ethyl acetate-methanol (5:5:1) as eluents to give 5 (0.16 g, 61%) as a white foam. ¹H-NMR (CDCl₃, 500 MHz): δ 1.87-1.94 (14H, m, SCH₂CH₂), 1.97, 2.04, 2.06, 2.15 (85H, 4s, 28 x CH₃CO), 2.58-2.87 (14H, m, SCH₂), 3.28 (7H, t, H-2), 3.34-3.37 (7H, m, H-4), 3.52-3.56 (7H, m, H-6^{a}), 3.67-3.84 (28H, m), 3.95-4.17 (28H, m), 4.52-4.58 (7H, m, H-1′), 4.78 (7H, s, OH), 4.90 (7H, s, H-1), 5.06 (7H, dd), 5.19 (7H, m, H-2′), 5.43 (7H, m, H-3′); ¹³C-NMR (CDCl₃, 125 MHz): δ 20.66, (28 x CH₃CO), 27.12 (SCH₂CH₂), 29.98 (SCH2), 51.45 (C-6), 60.37, 61.21, 67.20, 67.30, 68.0, 68.05, 70.35, 71.49, 71.78, 72.82, 74.25, 80.37, 84.50, 84.62, 01.65 (C-1), 169.54, 170.02, 170.19, 170.30, (28 x CH₃CO); MALDI m/z: 4164 [M+Na]⁺; Anal. Calcd for C₁₆₁H₂₃₁N₂₁O₉₁S₇: C, 46.70; H, 5.62; N, 7.10; Found: C, 46.44; H, 5.47; N, 6.96.

Heptakis[6-azido-6-deoxy-2-O-(3-(1′-thio-β-D-galactopyranosyl)propyl)]-β-cyclodextrin(6).

NaOMe-MeOH (1 M, 18 mL) was added to a stirred solution of 5 (1.8 g, 435 mmol) in dry MeOH (250 mL), and the reaction was allowed to stand at room temperature for 30 h. H₂O (30 mL) was added to dissolve precipitates, and the reaction was left to stir for a further 1 h, during which time the precipitate dissolved. The reaction was neutralized with Amberlite IR-120 (H+ form) ion-exchange resin and filtered. The solvents were removed in vacuo, and the resultant glass was dissolved in H₂O and freeze-dried to afford 6 as a white fluff(1.2 g, 93%). Selected NMR data. ¹H-NMR (D₂O, 300 MHz): δ 1.90-1.96 (14H, m, CH₂), 2.77-2.80 (7H, m, SCH₂), 4.47 (7H, d, H-′), 5.14 (7H, s, H-1); MALDI m/z: 2987 [M+Na]⁺.

Heptakis[6-amino-6-deoxy-2-O-(3-(1′-thio-β-D-galactopyranosyl)propyl)]-β-cyclodextrin(7).

6 (145 mg, 0.049 mmol) and triphenylphosphine (0.27 g, 1.03 mmol) were dissolved in DMF (3 mL) and the mixture was stirred at room temperature for 30 min. Aqueous ammonia (3 mL) was added and continued to stir at room temperature for 48 h. The mixture was diluted with methanol and evaporated in vacuo. The residue was dissolved in water (40 ml) and washed with chloroform until no UV active material was present in the organic layer. The aqueous layer was lyophilized to give 7 (121 mg,.88%) of as a solid. Selected NMR data. ¹H-NMR (D₂O, 300 MHz): δ 1.84-1.93 (14H, m, CH₂), 2.68-3.10 (14H, m, SCH₂), 4.46 (7H, d, H-1′), 5.19 (7H, s, H-1); ¹³C-NMR (D₂O, 75 MHz): δ 28.71, 28.76, 28.96, 43.52, 60.15, 60.9, 63.54, 70.37, 71.25, 71.62, 72.13, 72.68, 73.96, 74.7, 75.2, 76.44, 81.37, 82.17, 82.26, 82.29, 88.17, 88.3, 102.31; MALDI m/z: 2804 [M+Na]⁺.

Heptakis[6-amino-6-deoxy-2-O-(3-(1′-thio-β-D-galactopyranosyl)propyl)]-β-cyclodextrin acetate (8).

7 (86 mg, 0.031 mmol) was treated with aqueous HOAc (0.113 M, 1.92 mL), the solution was lyophilized to give 84 mg of 8 as a solid. Selected NMR data. ¹H-NMR (D₂O, 300 MHz): δ 1.84-2.14 (35H, m, CH₃CO and CH₂), 2.68-2.91 (14H, m, SCH₂), 4.47 (7H, d, H-1′), 5.27 (7H, s, H-1).

### EXAMPLE 5

### Reaction of Galactosyl-aminoCyD with NBD-X

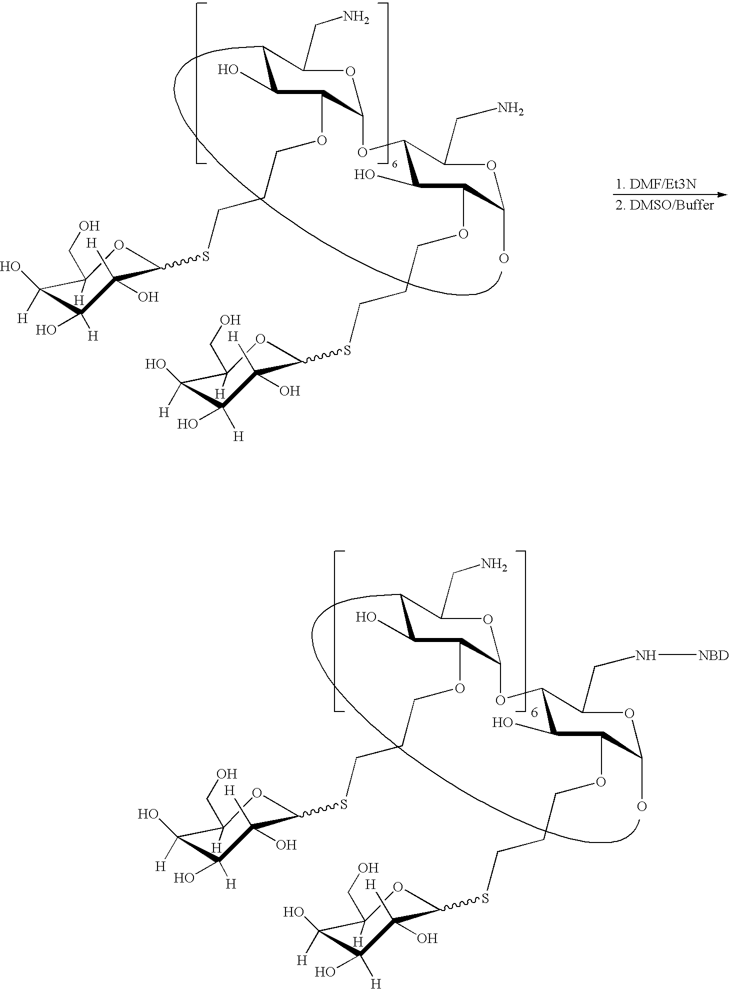

### EXAMPLE 6

### Generation of Galactose Cluster

Synthesis 2,2-Bis-hyroxymethyl-propane-1,3-diol mono tert-butyldiphenylsilyl ether

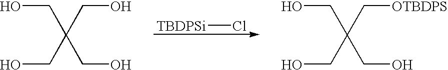

Mass Spectometry confirmed that it is 2,2-Bis-hyroxymethyl-propane-1,3-diol mono tert-butyldiphenylsilyl ether.

Synthesis of 2,3,4,6-tetra-O-acetyl-D-galactosyl bromide

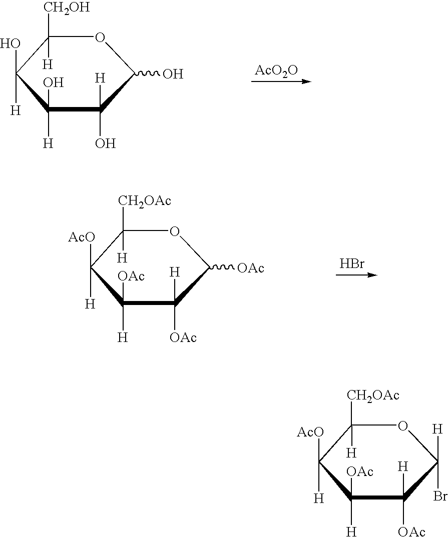

Pentaerythritol (8) was treated with tert-butyldiphenylsilyl chloride (TBDPSCl) in DMF at room temperature for 24 h to give the 2,2-Bis-hyroxymethyl-propane-1,3-diol mono tert-butyldiphenylsilyl ether(9), Which was converted into a 3-(2,3,4,6-O-tetraacety-galactopyranosy-oxymethyl)-2,2-Bis(2,3,4,6-O-tetraacety-galactopyranosy-oxymethyl)-propane-1-ol tert-butyldiphenylsilyl(10) ether by the reaction with Acetobromogalactose( 19) in present of AgClO₄/Ag₂CO₃ in CH₂Cl₂ at room temperature for 12 h. The desilylation reaction was carried out by tetrabutylammonium fluoride (TBAF) to give 3-(2,3,4,6-O-tetraacety-galactopyranosy-oxymethyl)-2,2-Bis(2,3,4,6-O-tetraacety-galactopyranosy-oxymethyl)-propanol(11) in 25% overall yield.

All compounds were confirmed by IR, MS and NMR spectroscopy. ˜40 g of acetobromogalactose.

Coupling reaction of acetobromogalactose with 2,2-Bis-hyroxymethyl-propane-1,3-diol mono tert-butyldiphenylsilyl ether

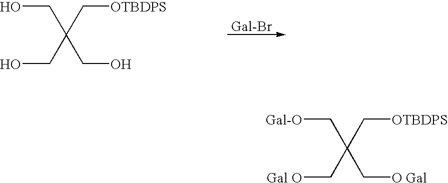

### EXAMPLE 7

### Generation of 6-Aminoglucose

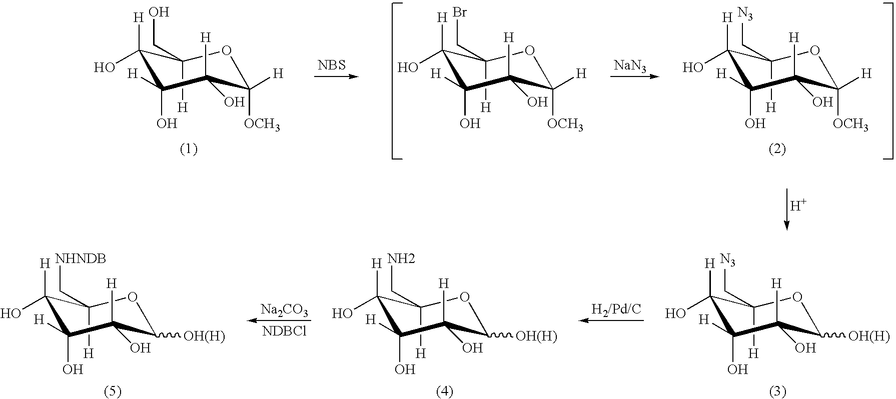

Methyl α-D-glucopyranoside (1) was purchased from Sigma-Aldrich Chemical Company Inc. All chemicals were reagent grade and were used without further purification unless otherwise noted.

Thin-layer chromatography (TLC) was carried out on Kieselgel 60 F254 (Merck) and visualization was accomplished by charring with 5% methanolic sulfuric acid. Column chromatography was performed using silica gel 60 (230-400) Mesh ASTM, 0.040˜0.063 mm, Rose Scientific Ltd. A Labconco Freeze Dryer 3 was used for lyophilization. IR spectra were recorded with a Perkin-Elmer 700 spectrometer. ¹H-NMR spectra and ¹³C-NMR spectra were recorded at Bruker AM-300. All chemical shifts are quoted in ppm, referenced to residual CHCl₃ at δ 7.27 for CDCl₃ solutions or HOD at δ 4.82 (25° C.) for D₂O solution Coupling constants (J) are reported in Hertz. ¹³C-NMR spectral assignments were aided by the J-MOD technique.

Methyl 6-azido-6-deoxy-a-glucopyranoside (2)

Methyl α-D-glucopyranoside (1) (13.0 g, 66.95 mmol) and Ph₃P (35.18 g, 133.8 mmol) were dissolved in dry DMF (280 mL). The mixture was cooled in an ice bath. NBS (24.1 , 135 mmol) was added and the mixture was stirred at 0° C. for 20 min. The ice bath was replaced with an oil bath and the mixture was heated at 55° C. for 3h. Methanol (15 mL) was added and the mixture was stirred for a further 10 min. Sodium azide (26.0 g, 400 mmol) was added and the mixture was heated at 85° C. for 4 h. The solvent was removed under vacuum and the residue was dissolved in water (250 mL). The aqueous phase was washed with methylene chloride (3×250 mL) and filtered. The aqueous solution was evaporated in vacuo and the residue was dried under high vacuum to yield a light yellow solid (11.6 g, 79% ); mp 45-47° C.; TLC (SiO₂) R_{f} 0.30 ( eluants 4:4:1 hexanes-ethyl acetate-methanol); IR (KBr) 2102.1 (N₃) cm⁻¹; ¹H-NMR (D₂O, 300 MHz) δ 4.81 (s, H-1), 3.43 (s, 3H, OCH₃); ¹³C-NMR (DMSO-d₆, 75 MHz) δ 99.8 (C-1), 72.9 (C-3), 71.7, 71.1(C-2, 5), 7.10 (C-4), 54.5 (OCH₃), 51.3 (C-6).

6-Azido-6-deoxy-D-glucopyranose (3)

2 (4.1 g, 18.7 mmol) was dissolved in 50 mL of water, Amberlite IR-120 (H⁺) (120 mL, wet volume) was added, and the mixture was heated under reflux for 7 h. The resin was filtered, and the filtrate was evaporated in vacuo. The residue was recrystallized from a mixture of 2-propanol and ether to yield a white solid (1.23 g, 32% ); mp 128-130° C.; TLC (SiO₂) R_{f} 0.17 (eluants 4:4:1 hexanes-ethyl acetate-methanol); ¹H-NMR (D₂O, 300 MHz) δ 5.20 (d, J_{1,2}=3.66 Hz, 1H, H-1α), 4.63 (d, J_{1,2}=7.93 Hz, 1H, H-1β); ¹³C-NMR (D₂O, 75 MHz) δ 98.4(C-1β), 94.6(C-1α), 78.0(C-3β), 76.9(C-3α), 76.6, 75.1, 73.9, 72.9(C-2α, 2β, 5α, 5β), 72.8(C-4β), 72.6(C-4α), 53.5, 53.4 (C-6α, C-6β.

6-Amino-6-deoxy-D-glucopyranose hydrochloride (4)

3 (0.5 g, 2.44 mmol) was dissolved in 120 mL of MeOH—H₂O (1:1), Pd/C (10%, 150 mg) was added, and the mixture was subjected to 30 psi of hydrogen at room temperature for 30 h. After removal of the catalyst by filtration, the solution was evaporated in vacuo to give colorless solid (0.47 g, 89%): mp 139˜141° C.; TLC (SiO₂) R_{f} 0.26 (eluants 7:2:2 isopropanol-water-ammonia water); ¹H-NMR (D₂O, 300 MHz) δ 5.13 (d, J_{1,2}=3.66 Hz, 1H, H-1α), 4.55 (d, J_{1,2}=7.94 Hz, 1H, H-1β); ¹³C-NMR (DMSO-d₆, 75 MHz) δ 97.1(C-1β), 92.4(C-1α), 76.1(C-3β), 74.6(C-3α), 72.4, 72.1, 71.9, 71.7(C-2α, 2β, 5α, 6β), 71.4(C-4β), 67.5(C-4α), 40.3 (C-6α, 6β).

6-Deoxy-6-(7-nitrobenzofuran-4-yl-amino)-D-glucopyranose (5)

4 (0.35 g, 1.62 mmol) was dissolved in 50 mL of MeOH—H2O (2:1), NBD-Cl (0.97 g, 4.87 mmol) and NaHCO₃ (2.05 g, 24.35 mmol) were added, and the mixture was stirred in the dark at room temperature for overnight. The solvent were removed in vacuo to a dark brown solid. The solid was purified by C18 reverse phase column chromatography using methanol-water as eluents for gradient elution. Evaporation of the appropriate fraction yielded a orange red solid (90 mg, 16%). mp>185° C. (decomp.); TLC (SiO₂) R_{f} 0.19 (eluants 9:3:1 chloroform-methanol-ammonia water); ¹H-NMR (DMSO-d₆, 300 MHz) δ 6.61 (d, J_{1,2}=2.66 Hz, 1H, H-1α), 6.34 (d, J_{1,2}=8.86 Hz, 1H, H-1β); ¹³C-NMR (DMSO-d₆, 75 MHz) δ 146, 145.9, 144.8, 144.2, 137.4, 137.2(C6H6), 96.9(C-1α), 92.3(C-1β), 76.4(C-3β), 74.7(C-3α), 73.9, 72.7, 72.5, 72.2(C-2α, 2β, 5α, 5β), 72.0(C-4β), 69.8(C-4α), 46.1(C-6β). 45.9(C-6α). ES-MS (in positive ionization) m/z calculated for C₁₂H₁₄N₄O₈ 343.27 (M+1), measured 343.1.

### EXAMPLE 8

### Characterization of AMP:BCD and AMP:aminoBCD Acetate Inclusion Complexes by QNMR

The interaction between BCD and AMP, and aminoBCD and AMP, was investigated to identify and quantify adenosine-5′-monophosphate(AMP) with β-cyclodexterin(CD) and 6-amino 1-cyclodextrin acetate(ACDA) inclusion complex using phosphorous NMR (P31) and differential scanning calorimetry (DSC).

A quantitative NMR method was adopted in order to measure the quantity of phosphor in AMP in inclusion complex with P31 NMR. The formation of inclusion complex between AMP and ACDA in which the sugar moiety of AMP is inserted into the hydrophobic cavity is supported by these chemical shifts of the sugar protons. The AMP and ACDA structure are listed below:

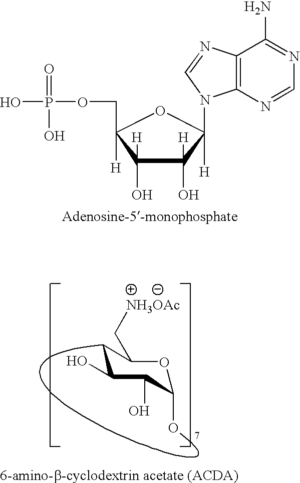

QNMR Instrumentation: P31 NMR experiments were carried out using a Eft-90 NMR spectrometer (Anasazi Instrument). Samples were dissolved in 0.5 mL of distilled water and spectra recorded using 5-mm NMR tubes. A typical set of acquisition parameters is listed in Table 1.Internal standards: Triethylphosphate (MW: 182, (C₂H₅)₃PO₄, William made) was selected and Potassium phosphate dibasic (K₂HPO₄) was used.

TABLE 1
Typical set of P31 NMR acquisition parameters
used for quantitative analyses

| Acquisition Parameter | Setting |
|---|---|
| SI-size | 65536 |
| NS-number of scans | 4 |
| RG-Rec. Gain | 50 |
| RD-Relaxation Delay | 200 sec. |
| AQ-Acquisition Time | 1.64 sec. |
| PW-Pulse Width | 16.3 usec. |
| FO-Field Offset | 1342 |
| Block | 5 |
| LB-Line Broading | 3.5 |

AMP 8.8 mg plus TEP 30 mg, AMP 17.5 mg plus TEP 30 mg, AMP 35 mg plus TEP 30 mg and AMP 70 mg plus TEP 30 mg were dissolved in 1 mL distilled water and filtered (0.22 um) then put 0.5 mL in NMR tube. AMP:BCD 1:1 (mole ratio) and AMP:ACDA 1:1 (mole ratio) inclusion complex were prepared by using solvent evaporation method. First, BCD or ACDA was dissolved in distilled water and AMP was added, sonicated, filtered (0.22 um) and then freeze dried.

To quantify the content of phosphorous by using optimum relaxation time, there are two methods: method A(using relaxation delay, RD=5˜7 times of the T1 value) and method B (block averaging with peak registration guide, BAPR). With BAPR method, we can get more accurate data. “INVREC” program and P31 list 2.txt (experiment 1 ˜8; 1.5, 3, 6, 12,24,48,96 and 192 sec) were used. As a pulse program, BAPR (ZGIG command) was used. The P31 spectrum of AMP/BCD and AMP/ACDA inclusion complex were acquired with T1 experiment condition, integral method and line fitting method.

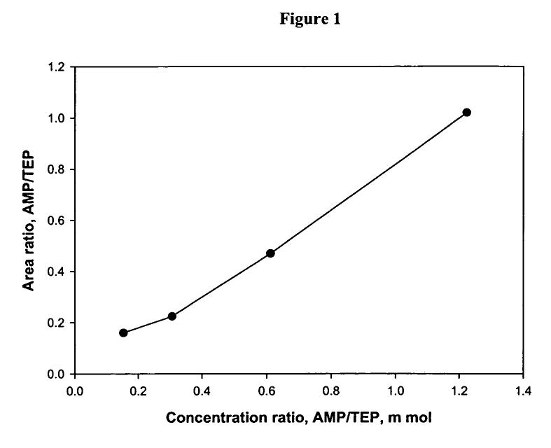

In P31 NMR spectroscopy, the signal intensity in a fully relaxed spectrum is directly proportion to the concentration: FIG. 1 which shows a standard curve of Y=0.8236X−0.0038, r2=0.9932. P31 NMR on AMP/BCD and AMP/ACDA inclusion complex. Integral method and line fitting method were used to characterize the AMP/BCD and AMP/ACDA inclusion complex.

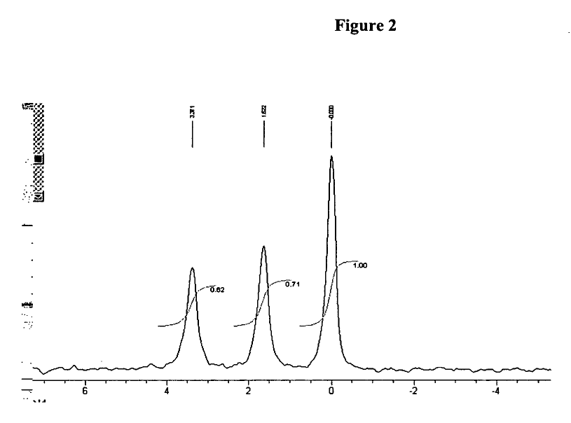

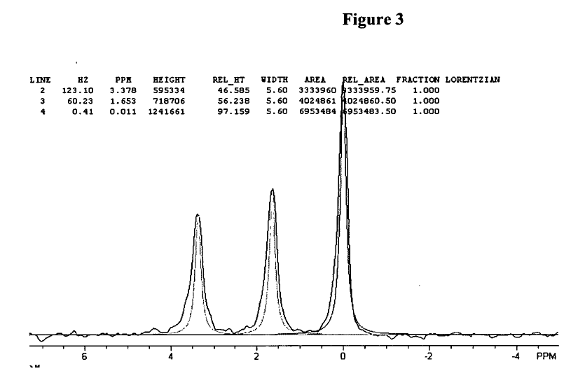

By using P31 NMR and with TEP internal standard, experimental conditions were set and standard curve was prepared the concentration range 8 mg to 70 mg AMP. As shown in FIG. 2 and FIG. 3; AMP/BCD 1:1 mole ratio (20 mg), dibasic Potassium phosphate (2.5 mg) and TEP(3 mg) in water(0.5 mL), three separate peaks were detected in P31 NMR with integral method and curve fitting method.

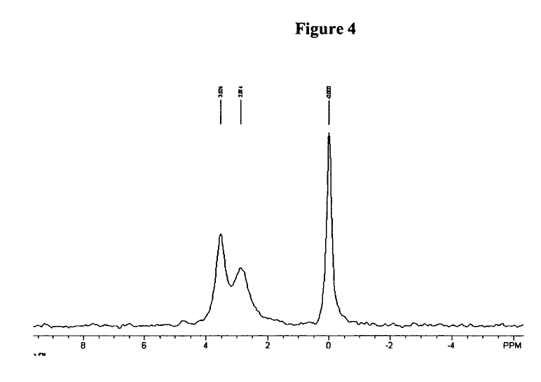

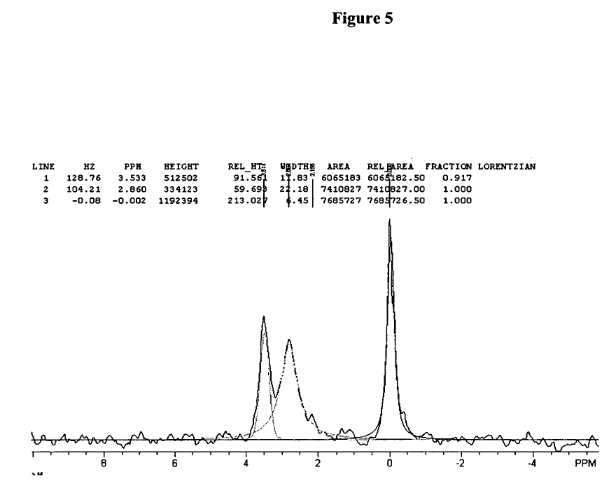

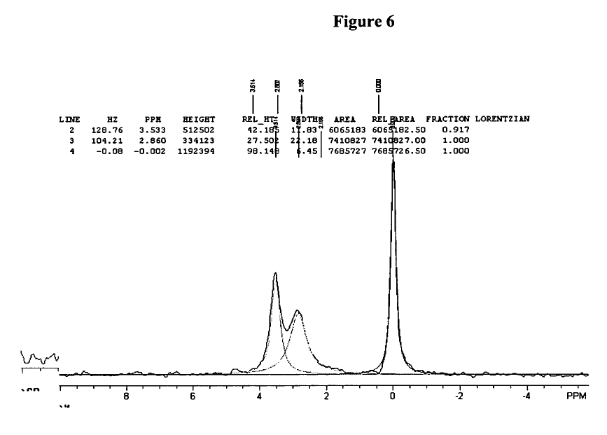

As shown in FIG. 4 and FIG. 5 (26 mg AMP:ABCDA 1:1 molar ratio) and in FIG. 6 (13 mg AMP:ABCDA 1:1 molar ratio); in the AMP/ACDA inclusion complex system, phosphate peak of Potassium phosphate was shifted to AMP, which indicated there is some interaction between AMP and ACDA. In the case of the AMP/ACDA 1:1 inclusion complex, the potassium phosphate peak and the AMP peak overlapped. This demonstrates an interaction between the phosphate of AMP and the amino's of ACDA.

Although an exact understanding of the interactions between the compounds of the present invention and nucleotides is not necessary to practise the present invention, it is proposed that phosphate groups of ADP and ATP are located at the primary hydroxyl side in the complexes and adenines are located at the secondary hydroxyl side, and that the sugar moiety is located in the cavity of monoamino derivative of β-CD.

### EXAMPLE 9

### Characterization of AMP:BCD and AMP:aminoBCD Acetate Inclusion Complexes by DCA

AMP:BCD 1:1(mole ratio) and AMP:ACDA 1:1 (mole ratio) inclusion complex were prepared by using solvent evaporation method. First, BCD or ACDA was dissolved in distilled water and AMP was added, sonicated, filtered (0.22 um) and then freeze dried.

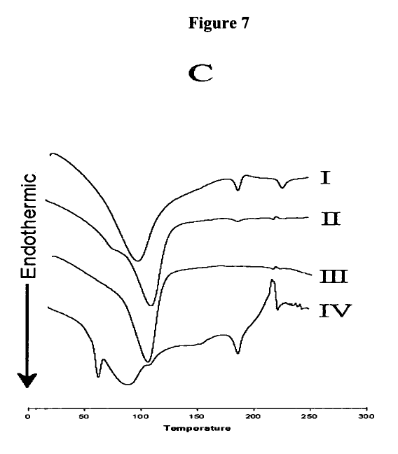

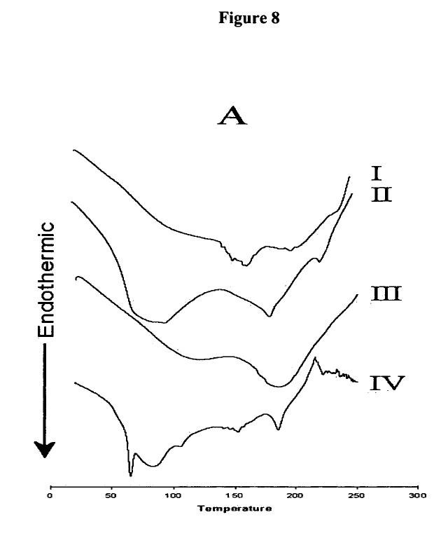

DSC 120 (SEIKO SII, model SSC/5200) was used to trace the DSC profiles of BCD, ACDA, AMP, AMP/BCD or AMP/ACDA inclusion complex and physical mixture. DSC thermograms were recorded by placing weighed quantities (3-5 mg) in a sealed aluminium pan. Scans were performed between 20° and 250° C. with heating rate 10° C./min. Inclusion complex were made solvent evaporation method and physical mixtures were prepared in same molar ratios by gently blending in an AMP with BCD or ACDA. FIG. 7 shows the result of a DSC scan on AMP/BCD and FIG. 8 on AMP/ACDA inclusion complex

The thermal behaviour of AMP/BCD inclusion complex, physical mixture, and AMP/ACDA inclusion complex, physical mixture are reported in these figures. β-CD eliminated water in the around 100° C. with a broad endothermic effect. In AMP/BCD inclusion complex DSC, at 180° C., a small endothermic peak can be found, which is originated from AMP.

In AMP/ACDA inclusion complex DSC, around 160° C. a broad endothermic effect is detected. Also, no endothermic peak is found at 180° C. These changed patterns indicate some interaction between AMP and ACDA.

### EXAMPLE 10

### Complexation of Poly-araA (PA) with Heptakis[6-amino-6-deoxy-2-O-(3-(1′-thio-β-D-galactopyranosyl)propyl)]-β-cyclodextrin acetate (GCDA)

An oligonucleotide araA-monophosphate (araAMP) 12-mer was complexed with GCDA in a 1:1 ratio. As determined using zone capilliary electrophoresis, the PA:GCDA resulted in a complexation of 10% of the PA with GCDA. At 2:1, PA:GCDA complexation reaches 75%. Addition of 0.01M Sodium tetraborate buffer results in disassociation of the 2:1 ratio complex, with only 45% complexed after 4 hours. Increasing the buffer to 0.08M Sodium tetraborate results in faster disassociation of the complex. The complex in water is stable with no decrease in complexation resulting after 24 hours in unbuffered water. It is therefore concluded that a charge association complex is formed between the GCDA and PA.

### EXAMPLE 11

### Protection of Poly-araA Through Complexing with GCDA

100 ng/μL of PA and PA-GCDA 250 ng/μL(as PA 100 ng/μL) in water were prepared and kept in −20° C.

100 μL of fresh rat plasma was mixed with the PA or PA-GCDA and incubated at 37° C. in a water bath for 10 min, 30 min, or I hr. The samples were stored at −20° C. until HPLC testing.

HPLC conditions were as listed in Table 2

TABLE 2
HPLC Conditions.

| | |
|---|---|
| Mobile phase: | A: 0.1 M triethylammonium acetate |
| | buffer, pH 7.0 |
| | B: Acetonitrile |
| Gradient: | B 3% to 15% in 30 min. |
| Column: | Radial-Pak C18, 4 u |
| Flow: | 1.0 mL/min |
| Injection: | 10 uL |
| Detection: | UV 260 nm |

Degradation of the polynucleotide, and the existence of a PA-GCDA complex, 10 was observed through resolution of peaks through HPLC. Degradation products were observed at approximately 7 min of HPLC run-time with both PA and PA-GCDA. Both PA and PA-GCDA complex demonstrated similar degradation profiles. The kinetics of degradation on PA-GCDA complex was observed to be slower than that of PA alone.

### EXAMPLE 12

### Anti-viral Activity with CGDA:PA and GCDA:araAMP Complexes

Tetracycline-responsive cell lines HepAD38 were used (Ladner et al., 1997), hepatoma cells that have been stably transfected with a cDNA copy of the pregenomic RNA of wild-type virus. Withdrawal of tetracycline from the culture medium results in the initiation of viral replication. Cells were cultured at 37° C. in a humidified 5% CO₂/air atmosphere in seeding medium, DMEM/Ham's F12 (50/50) supplemented with 10% (v/v) heat-inactivated fetal calf serum, 100 IU/ml penicillin/50 μg/ml streptomycin mix, 400 μg/ml G418, and 0.3 μg/ml tetracycline.

Upon assay initiation, the cells were seeded in 48-well plates at a density of 1.5×10⁵/well. After 2-3 days the cultures were induced for viral production by washing with prewarmed PBS and were fed with 200 μl assay medium (seeding medium without tetracycline and G418) with or without the antiviral compounds. Medium was changed after 3 days. The antiviral effect was quantified by measuring levels of viral DNA [isolated (Qiagen) from the cell cultures] at day 4 post-induction, by a real time quantitative PCR (Q-PCR) and analyzed using a SDS 7000 (Applied Biosystems, Foster City, Calif.).

A plasmid containing the full length insert of the HBV genome was used to prepare the standard curve. The amount of viral DNA produced in treated cultures was expressed as a percentage of the mock treated samples. The cytostatic effect of the various compounds was assessed employing the parent hepatoma cell line HepG2. The effect of the compounds on exponentially growing HepG2 cells was evaluated by means of the MTS method (Promega). Briefly, cells were seeded at a density of 3000/well (96 well plate) and were allowed to proliferate for 3 days in the absence or presence of compounds after which time cell density was determined.

As can be seen in Table 3, the use of GCDA increased the therapeutic efficacy of the nucleoside analogue araA. Furthermore, the utility of PA as an anti-viral compound is demonstrated when used in combination with GCDA.

TABLE 3
Improvement of Therapeutic effect using GCDA

| Compound | EC₅₀ (μg/ml) | Improvement Compared to araA |
|---|---|---|
| poly-araA (PA) | 29 | 1.67 |
| araA | 35 | 1.00 |
| araAMP | 33 | 1.35 |
| PA-GCDA | 29 | 2.92 |
| araA-GCDA | 316 | 2.43 |
| araAMP-GCDA | 141 | 5.45 |

Although the above disclosure describes and illustrates various embodiments of the present invention, it is to be understood that the invention is not to be limited to these particular embodiments. Many variations and modifications will now occur to those skilled in the art. For a full definition of the scope of the invention, reference is to be made to the appended claims.

What is claimed is:

1. A compound useful for delivery of a drug to mammalian cells comprising the general formula: 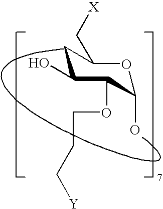
wherein X is a molecule of net positive or negative charge at physiologic conditions, and Y is a molecule selected from the group consisting of carbohydrates and polypeptides and wherein Y is capable of enhancing intracellular delivery of the compounds of the present invention to mammalian cells.

2. The compound of claim 1 wherein the compound useful for delivery of a drug to mammalian cells comprising the general formula:

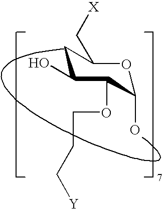

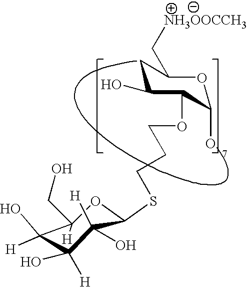

3. A composition useful for treatment of viral infection of a mammalian cell comprising a complex formed between a compound of the general formula of: 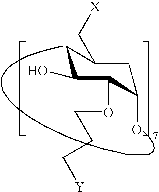
wherein X is a molecule of net positive or negative charge at physiologic conditions, and Y is a molecule selected from the group consisting of carbohydrates and polypeptides and wherein Y is capable of enhancing intracellular delivery of the compounds of the present invention to mammalian cells; and
Bₙ
wherein B is a monomeric or oligomeric nucleotide analogue where n=1,2,3,4, . . . 50.

4. The composition of claim 3 wherein Bₙ is an oligonucleotide araA-monophosphate (araAMP) with n=12.

5. The composition of claim 3 wherein B, is a monomeric araAMP.

6. The composition of claim 3 wherein the compound of the general formula of:

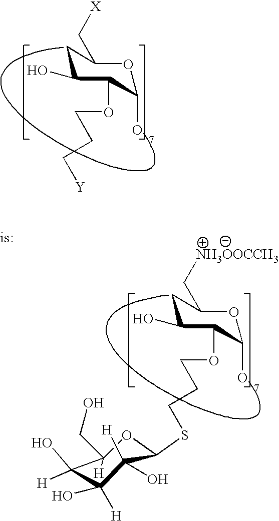

7. The composition of claim 4 wherein the compound of the general formula of:

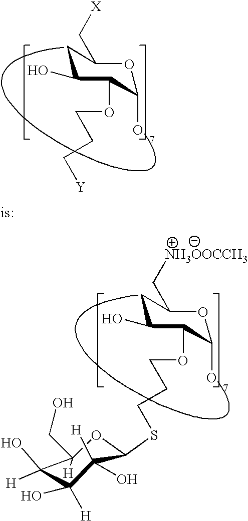

8. The composition of claims 5 wherein the compound of the general formula of: [chemical structure]
